(19) United States
(12) Patent Application Publication
(10) Pub. No.: US 2010/0158831 A1
(43) Pub. Date: Jun. 24, 2010

(54) Viscosity Regulator, Method For The Production Thereof, And Use Thereof

(76) Inventors: Bert Volkert, Berlin (DE); André Lehmann, Potsdam (DE); Heiko Nerenz, Hoxter (DE); Andreas Achrader, Bevern (DE)

Correspondence Address: GIBSON & DERNIER LLP, 900 ROUTE 9 NORTH, SUITE 504, WOODBRIDGE, NJ, 07095, US

(21) Appl. No.: 12/593,627

(22) PCT Filed: Mar. 28, 2008
(86) PCT No.: PCT/EP08/02492

§ 371 Date: Feb. 5, 2010

(30) Foreign Application Priority Data
Mar. 29, 2007 (DE) 10-2007-015-282.7

(51) Int. Cl. A61K 47/36; A61K 31/045; A61K 31/047; A61K 36/00; A61K 8/73; C08B 31/10; A61Q 17/04

(52) U.S. Cl. 424/59; 514/729; 514/738; 424/725; 536/111

(57) ABSTRACT

The present invention relates to a method for the production of a viscosity regulator by introducing hydrophobic and hydrophilic groups into a starch molecule. The invention also relates to viscosity regulators produced in this manner and the use thereof. The viscosity regulators according to the invention can be used as emulsifiers or as thickening agents of surfactant-containing systems, e.g. in cosmetic or pharmaceutical preparations.

The invention relates to a method for the production of a viscosity regulator by introducing hydrophobic and hydrophilic groups into a starch molecule. The invention also relates to viscosity regulators produced in this manner and the use thereof. The viscosity regulators according to the invention can be used as emulsifiers or as thickening agents of surfactant-containing systems, e.g. in cosmetic or pharmaceutical preparations.

Thickening agents represent important aids in cosmetics and in pharmaceutics in order to adjust, in different preparations, such as e.g. emulsions or surfactant cleaning products, a suitable consistency, i.e. viscosity.

Specific commercial products, such as e.g. hydroxyethyl cellulose, exhibit a slow enzymatically caused viscosity reduction in the end product. Corresponding enzymes can be introduced with specific raw materials into the end product.

The overall goal is synthesis of starch-based hydrophilically-hydrophobically modified thickening agents for surfactant-containing products based on renewable raw materials. The corresponding products are intended to be toxicologically safe, stable in the long term and surfactant-compatible and to enable the highest possible transparency of the surfactant end product.

There can be used as thickening agents for cosmetic and pharmaceutical preparations compounds from different substance classes. Above all, PEG derivatives are used as thickening agents, which are evaluated critically more and more frequently with respect to toxicology. Thickening agents are used in various products in order to adjust a suitable consistency, i.e. viscosity (K. Schrader, Grundlagen und Rezepturen der Kosmetika, 3^{rd} edition, Hüthig, Heidelberg 2005; W. Umnbach, Kosmetik und Hygiene, 3^{rd} edition, Wiley-VCH, Weinheim, 2004). Not only is the product viscosity affected but also the flow behaviour as a function of external shear forces. In addition to inorganic substances, such as polysilicic acids or montmorillonites, these are above all organic polymers (E. D. Goddard, J. V. Gruber, Principles of Polymer Science and Technology in Cosmetics and Personal Care, Marcel Dekker Inc., New York, 1999). These can be divided into natural thickeners (e.g. starch, gelatine, alginates), modified natural substances, (e.g. cellulose ether, hydroxyethyl- and -propyl cellulose) and also totally synthetic polymers (e.g. polyacrylates, polyamides, polyethers) (J. Falbe, M. Regitz, Römpp Chemielexikon, Thieme 1995). There is thereby no universally useable thickening agent since, according to the substance class, properties, such as low electrolyte compatibility, low transparency of the gel, low long term stability or unsuitability on sensory grounds, preclude use in specific product groups.

In cosmetics, various natural starches, from e.g. potatoes, maize or rice, are used. Furthermore, the following starch derivatives are used (descriptions according to INCI; functionalities):
ALUMINIUM STARCH OCTENYLSUCCINATE absorption agents/viscosity regulators
CORN STARCH/ACRYLAMIDE/SODIUM ACRYLATE COPOLYMER antistatic agents/combing aids/film formers
DISTARCH PHOSPHATE binders
DISTARCH GLYCERYL ETHER binders
GLYCERYL STARCH absorption agents/binders
HYDROGENATED STARCH HYDROLYSATE moisturisers
HYDROLYSED CORN STARCH binders/viscosity regulators
HYDROLYSED SOY STARCH skin conditioner
HYDROLYSED WHEAT STARCH viscosity regulators
HYDROXYPROPYL CORN STARCH viscosity regulators
SODIUM CARBOXYMETHYL STARCH emulsion stabiliser, film formers
SODIUM HYDROXYPROPYL STARCH PHOSPHATE viscosity regulators
SODIUM POLYACRYLATE STARCH film formers
SODIUM STARCH OCTENYLSUCCINATE, absorption agents/viscosity regulators
STARCH DIETHYLAMINOETHYL ETHER
antistatics/combing aids/film formers
STARCH/ACRYLATES/ACRYLAMIDE COPOLYMER binders/film formers.

Of the listed starch derivatives, none is suited optimally for use as surfactant thickener. The mentioned low-substituted sodium carboxymethyl starch exhibits a highly thickening effect in aqueous systems but leads to turbid products and is not sufficiently electrolyte-stable.

Specifically non-ionic alkylpolyglycosides which are classified from a toxicological point of view as safe can only be thickened with great difficulty.

Starting herefrom, it was the object of the present invention to provide viscosity regulators which exhibit good properties as thickener in surfactant formulations and at the same time lead to low turbidity.

This object is achieved by the method having the features of claim 1 and the viscosity regulator having the features of claim 14. Likewise, emulsions having the features of claim 11 are provided according to the invention. Uses according to the invention are listed in claims 15 and 23. The further dependent claims reveal advantageous developments.

According to the invention, a method for the production of a viscosity regulator is provided, in which firstly starch is modified hydrophobically by alkylation and/or hydroxyalkylation and, in a subsequent step, in addition hydrophilic groups are introduced by carboxymethylation with a halogenated acid.

According to the invention, viscosities and turbidities in surfactant-containing cleaning products which contain this viscosity regulator can thus be adjusted. For stabilisation of the viscosity of such surfactant systems, the double-substitution of the carboxymethyl starch is required in order thus to enable hydrophilic and hydrophobic interactions of the starch derivative with the surfactant. The ratio of hydrophilic and hydrophobic functional groups of the starch derivative can be achieved by carboxymethylation for the hydrophilic groups and by alkylation or hydroxyalkylation for the hydrophobic groups.

The sequence of the substitution of the starch hereby plays a crucial role for the viscosity and turbidity of surfactant systems in which the viscosity regulator according to the invention is used as thickening agent. Thus only starch derivatives in which firstly an alkylation or hydroxyalkylation of the starch and subsequently a carboxymethylation were effected were able according to the invention to have the required properties as viscosity regulator.

Preferably, the alkylation is implemented with a linear or branched C₁-C₂₀ alkylhalogenide, linear or branched C₁-C₁₂ alkylhalogenides being particularly preferred.

The alkylation is thereby preferably implemented at temperatures in the range of 40° C. to 100° C., particularly preferred in the range of 45° C. to 80° C.

The alkylation of the starch is thereby implemented preferably such that the degree of substitution (DS) with respect to the alkyl substituents is in the range of 0.1 to 1.2 and the degree of substitution with respect to the carboxymethyl substituents is in the range of ≧0 to 1.2.

Another preferred variant provides that the introduction of hydrophobic groups is effected by a hydroxyalkylation with a linear or branched 1,2-epoxyalkane, the alkane preferably having a chain length of 6 to 12 C-atoms. Linear or branched 1,2-epoxyalkanes are hereby used for particular preference, the alkane having a chain length of 6 to 12 C-atoms.

Preferably, the hydroxyalkylation is implemented at temperatures in the range of 80° to 160° C., particularly preferred at temperatures in the range of 100° to 150° C.

In the case of hydroxyalkylation, the degree of substitution (DS) with respect to the hydroxyalkyl substituents is preferably in the range of 0.1 to 1.2, whilst the degree of substitution with respect to the carboxycarbyl substituents is in the range of 0.5 to 1.6.

Preferably, the halogenated acid is selected from the group comprising monochloroacetic acid, monochloropropionic acid, chloromalonic acid and mixtures hereof.

The starch is selected preferably from the group comprising potato, wheat-, rice-, maize-, barley-, tapioca starch and mixtures hereof.

With respect to the method for the carboxymethylation of starch, reference is made explicitly to DE 100 33 197 C1 and the method conditions indicated here, e.g. the solvent.

According to the invention a viscosity regulator which was produced according to the above-described method is likewise provided.

The above-described viscosity regulator is used for thickening surfactant-containing preparations for cosmetic or pharmaceutical application. In a preferred embodiment, a surfactant-containing preparation of this type has sodium lauryl ether sulphate which is contained particularly preferably in a concentration of 0.5 to 3% by weight, relative to the total preparation. However, the sodium lauryl ether sulphate thereby causes significantly clearer emulsions which have a higher viscosity.

The viscosity regulator in surfactant-containing cleaning products, e.g. shower gels or shampoos, is used for particular preference. Two examples of surfactant-containing cleaning products according to the invention are listed in Table 1.

TABLE 1

| | | % | % |
|---|---|---|---|
| Pos. | Raw material | [w/w] | [w/w] |
| 1 | Water | 83.650 | 83.100 |
| 2 | LAURYL GLUCOSIDE | 12.000 | 14.000 |
| 3 | SODIUM LAURETH SULPHATE | 2.500 | 1.000 |
| 4 | 2-hydroxyoctylcarboxymethyl starch | 0.500 | 0.600 |
| 2 | Edeta BD (Trilon BD) | 0.050 | 0.100 |
| 3 | Citric acid | q.s. | q.s. |
| 4 | Preservatives | 1.000 | 1.000 |
| 5 | POLYQUATERNIUM-7 | 0.100 | — |
| 6 | PARFUM | 0.200 | 0.200 |
| | | 100.000 | 100.000 |

According to the invention, an oil-in-water emulsion is likewise provided, which contains an oil component, a lipophilic or hydrophilic active ingredient and at least one viscosity regulator as emulsifier, as was described previously.

The emulsion thereby contains preferably 0.1 to 5.0% by weight of the viscosity regulator, relative to the total emulsion.

A preferred variant provides that the emulsion contains alkylpolyglycosides which are contained for particular preference in a concentration of 0.5 to 20% by weight, in particular 1 to 15% by weight, relative to the total emulsion.

The emulsion is present preferably in the form of a cream or lotion.

A further preferred embodiment provides that the emulsion is free of low-molecular ionic or hydrophilic, non-ionic emulsifiers, as are used normally, and consequently display good care characteristics on the skin.

The lipophilic active ingredients contained in the emulsion are preferably selected from the group consisting of vitamins, oil-soluble UV filters, bisabolol, fragrances and mixtures hereof.

The hydrophilic active ingredients are preferably selected from the group consisting of polyols, e.g. glycerine or sorbitol, urea, plant extracts, vitamins, self-tanning compounds, e.g. dihydroxyacetone, UV filters or mixtures hereof.

The emulsion preferably contains an oil component selected from the group consisting of vegetable oils, e.g. soya oil, olive oil or almond oil, paraffin oils, di-N-alkylether, fatty acids, fatty alcohols, ester oils, natural and synthetic waxes, silicone compounds and mixtures hereof.

Furthermore, the emulsion can contain additives selected from the group consisting of preservatives, solvents, such as e.g. alcohols or glycols, antioxidants, fillers, hydrocolloid formers, such as e.g. xanthan gum, pigments, chelating agents, such as EDTA, pH regulators, e.g. citric acid, and mixtures hereof.

In the subsequent Table 2, compositions of O/W emulsions according to the invention are listed.

TABLE 2

| | | % | % | % |
|---|---|---|---|---|
| Pos. | Raw material | [w/w] | [w/w] | [w/w] |
| 1 | CAPRYLIC/CAPRIC | 10.000 | 10.000 | 10.000 |
| | TRIGLYCERIDE | | | |
| 2 | DECYL OLEATE | 10.000 | 10.000 | 5.000 |
| 3 | Soya oil | — | — | 5.000 |
| 4 | CETEARYL ALCOHOL | 3.000 | 3.000 | 3.000 |
| 5 | GLYCERYL STEARATE | 2.000 | 2.000 | 2.000 |
| 6 | Water | 67.900 | 65.700 | 66.900 |
| 7 | GLYCERINE | 5.000 | 5.000 | 5.000 |
| 8 | DISODIUM EDTA | 0.100 | 0.100 | 0.100 |
| 9 | Preservatives | 1.000 | 1.000 | 1.000 |
| 10 | Xanthan gum | — | 0.200 | — |
| 11 | 2-hydroxyoctylcarboxymethyl | 1.000 | 3.000 | 2.000 |
| | starch | | | |
| | | 100.000 | 100.000 | 100.000 |

The subject according to the invention is intended to be explained in more detail with reference to the subsequent examples without wishing to restrict said subject to the special embodiments shown here.

### EXAMPLE 1

### Preparation of 2-Hydroxyoctyl Starch DS_{2-hydroxyoctyl}=0.7

9.6 g sodium hydroxide and 34.089 water-free sodium sulphate are dissolved in 640 ml distilled water and placed in a suitable pressure reactor. 77.9 g (dry) waxy maize starch are added to the alkaline solution and agitated. After 30 min, 73.84 g 1,2-epoxyoctane are added and heated to 140° C. and this temperature is maintained for 3.5 h, a pressure of 2.5 bar being set. Hereafter, the reactor is cooled and the raw product is comminuted firstly with an Ultra Turrax before the pH value is adjusted neutrally with 16% hydrochloric acid. The product is washed with distilled water and dried in air. A coarse-grained 2-hydroxyoctyl starch is obtained, which is water-insoluble and has a degree of substitution of 0.7.

### EXAMPLE 2

### Preparation of 2-Hydroxyoctylcarboxymethyl Starch DS_{carboxymethyl}=0.92

280 ml 2-propanol are placed in a suitable reactor and 35.0 g monochloroacetic acid are dissolved with constant agitation. 14.8 g sodium hydroxide are added and, after thorough mixing, 27.0 g (dry) 2-hydroxyoctyl starch (DS_{2-hydroxyoctyl}=0.7) are added. Subsequently, 14.8 g sodium hydroxide are added again and heated to 40° C. The temperature is maintained for 4.5 h. After cooling of the reactor, the pH value of the raw product is neutralised with 50% propanolic acetic acid. The product was processed by means of dialysis. 2-hydroxyoctylcarboxymethyl starch is obtained which is clear and completely soluble in water. The degree of substitution is DS_{carboxymethyl}=0.92.

### EXAMPLE 3

### Preparation of Hydroxyoctylcarboxymethyl Starch DS_{carboxymethyl}=0.77

280 ml 2-propanol are placed in a suitable reactor and 29.2 g monochloroacetic acid are dissolved with constant agitation. 12.3 g sodium hydroxide are added and, after thorough mixing, 263 g (dry) 2-hydroxyoctyl starch (DS_{2-hydroxyoctyl}=0.7) are added. Subsequently, 12.3 g sodium hydroxide are added again and heated to 40° C. The temperature is maintained for 4.5 h. After cooling of the reactor, the raw product is absorbed in methanol and the pH value is neutralised with 50% propanolic acetic acid. By processing the product by means of dialysis, 2-hydroxyoctylcarboxymethyl starch is obtained, which is clear and completely soluble in water. The degree of substitution is DS_{carboxymethyl}=0.77.

### EXAMPLE 4

### Preparation of 2-Hydroxydecyl Starch DS_{2-hydroxydecyl}=0.9

9.6 g sodium hydroxide and 34.08 g water-free sodium sulphate are dissolved in 640 ml distilled water and placed in a suitable pressure reactor. 77.8 g (dry) waxy maize starch are added to the alkaline solution and agitated. After 30 min, 89.9 g 112-epoxydecane is added and heated to 140° C. and this temperature is maintained for 3.5 h, a pressure of 3 bar being set. Hereafter, the reactor is cooled and the raw product is comminuted firstly with an Ultra Turrax before the pH value is adjusted neutrally with 16% hydrochloric acid. The product was washed with distilled water and dried in air. A coarse-grained 2-hydroxydecyl starch is obtained, which is water-insoluble and has a degree of substitution of 0.9.

### EXAMPLE 5

### Preparation of 2-Hydroxydecylcarboxymethyl Starch DS_{carboxymethyl}=0.84

150 ml 2-propanol are placed in a suitable reactor and 33 g monochloroacetic acid are dissolved with constant agitation. 14.0 g sodium hydroxide are added and, after thorough mixing, 19.6 g (dry) 2-hydroxydecyl starch (DS_{2-hydroxydecyl}=0.9) are added. Subsequently 14.0 g sodium hydroxide are added again and heated to 40° C. The temperature was maintained for 4.5 h. After cooling of the reactor, the raw product is absorbed in methanol and the pH value is neutralised with 50% propanolic acetic acid. The product cleaned by means of dialysis produces 2-hydroxydecylcarboxymethyl starch which is clearly water-soluble and with a degree of substitution of DSC_{carboxymethyl}=0.84.

### EXAMPLE 6

### Preparation of 2-Hydroxydodecyl Starch DS_{2-hydroxydodecyl}=0.9

9.6 g sodium hydroxide and 34.08 g water-free sodium sulphate are dissolved in 540 ml distilled water and placed in a suitable pressure reactor. 77.9 g (dry) waxy maize starch are added to the alkaline solution and agitated. After 30 min, 106.2 g 1,2-epoxydodecane are added and heated to 140° C. and this temperature is maintained for 3.5 h, a pressure of 3.5 bar being set. Hereafter, the reactor is cooled and the pH value is adjusted neutrally with 16% hydrochloric acid. The product is washed with distilled water and dried in air. A coarse-grained 2-hydroxydodecyl starch is obtained, which is water-insoluble and has a degree of substitution of 0.9.

### EXAMPLE 7

### Preparation of 2-Hydroxydodecylcarboxymethyl Starch DS_{carboxymethyl}=0.65

34 g monochloroacetic acid are placed in 150 ml 2-propanol in a suitable reactor and dissolved with constant agitation. 14.2 g sodium hydroxide are added and, after thorough mixing, 20 g (dry) 2-hydroxydodecyl starch (DS_{2-hydroxydodecyl}=0.9) are added. Subsequently 14.2 g sodium hydroxide are added again and heated to 40° C. The temperature is maintained for 4.5 h. After cooling of the reactor, the raw product is absorbed in methanol and the pH value is neutralised with 50% propanolic acetic acid. By processing by means of dialysis, 2-hydroxydecylcarboxymethyl starch is obtained, which is turbidly soluble in water and has a degree of substitution of DS_{carboxymethyl}=0.65.

### EXAMPLE 8

### Preparation of Methyl Starch DS_{methyl}=0.73

Firstly, 150 g (dry) waxy maize starch are suspended with 1 1 2-propanol in a suitable agitation vessel and mixed with 222 g sodium hydroxide. After addition of 20 ml water, the reaction batch is agitated for a further 2 h at room temperature.

The alkaline waxy maize starch is absorbed in 2 1 2-propanol and the solvent is distilled off.

250 g of this alkali starch together with 400 ml 2-propanol, 23 ml methanol and 31 ml water are placed in a suitable pressure reactor and cooled to −10° C. 50 ml methyl chloride are added to the pressure reactor. Subsequently, heating takes place for 8 h at 67° C. The reactor is cooled to room temperature, after which the product is neutralised with 2-propanol/acetic acid (1:1).

The product cleaned by means of dialysis produces methyl starch which is clearly soluble in water and has a degree of substitution of DS_{methyl}=0.73.

### EXAMPLE 9

### Preparation of Methylcarboxymethyl Starch DS_{carboxymethyl}=0.43

180 ml 2-propanol are placed in a suitable reactor and 19 g monochloroacetic acid is dissolved with constant agitation. 16.0 g sodium hydroxide are added and, after thorough mixing, 19.2 g (dry) methyl starch from example 8 are added. Subsequently, 8.0 g sodium hydroxide are added again and heated to 40° C. The temperature is maintained for 4.5 h. After cooling of the reactor, the raw product is absorbed in methanol and the pH value is neutralised with 50% propanolic acetic acid. The product cleaned by means of dialysis produces methylcarboxymethyl starch which is turbidly soluble in water and has a degree of substitution of DS_{carboxyl}=0.43.

### EXAMPLE 10

### Starch Derivatives in Alkylpolyglycoside-Containing Solutions

For examination of the viscosity and the turbidity, a solution containing 14% alkylpolyglycoside and 0.5% 2-hydroxyoctylcarboxymethyl starch was produced. For this purpose, 0.36 g (dry) of this starch derivative synthesised in example 2 were dissolved in 51.8 g distilled water and added to 21.4 g alkylpolyglycoside. The thus produced solution is kept in motion for 2 d on a roller mixer.

The measurements of viscosity and turbidities produced a shear viscosity of 7540 mPas with a shear rate of γ=2.55 s⁻¹, and also a light transmission of 60%.

### EXAMPLE 11

### Starch Derivatives in Alkylpolyglycoside-Containing Solutions

For examination of the viscosity and the turbidity, a solution containing 14% alkylpolyglycoside and 0.5% 2-hydroxyoctylcarboxymethyl starch was produced. The 2-hydroxyoctylcarboxymethyl starch used here was synthesised in that the starch was firstly carboxymethylated and subsequently alkylated.

For the surfactant solution, 0.36 g (dry) starch derivative were dissolved in 51.8 g distilled water and added to 21.4 g of the alkylpolyglycoside. The thus produced solution is kept in motion for 2 d on a roller mixer.

The measurements of the viscosity and turbidities produced a shear viscosity of 4315 mPas at a shear rate of γ=2.55 s⁻¹, and also a light transmission of 3%.

### EXAMPLE 12

### Starch Derivatives in Solutions with Surfactant Mixtures

For examination of the viscosity and the turbidity, a solution containing 10% alkylpolyglycoside, 2.25% sodium lauryl ether sulphate and 1% 2-hydroxyoctylcarboxymethyl starch was produced. For this purpose, 0.76 g (TG: 92.2%) of the starch derivative synthesised in example 2 were dissolved in 51.9 g distilled water and added to a mixture of 15 g alkylpolyglycoside and 6 g sodium lauryl ether sulphate.

The thus produced solution is kept in motion for 2 d on a roller mixer. The measurements of the viscosity and turbidities produced a shear viscosity of 32800 mPas at a shear rate of γ=2.55 s⁻¹, and also a light transmission of 92%.

Table 3 and 4 show overviews of the transmission and shear viscosities of selected starch derivatives in various surfactant systems (SD=starch derivative; CMS=carboxymethyl starch; APG=alkylpolyglycoside; SLES=sodium lauryl ether sulphate).

TABLE 3

| | | | 2-hydroxy- | 2-hydroxy- | 2-hydroxy- |
|---|---|---|---|---|---|
| | | | octyl-CMS | decyl-CMS | dodecyl-CMS |
| | | Ex. | 2 | 5 | 7 |
| | | DS_{carboxymethyl} | 0.7 | 0.9 | 0.9 |
| | | DS_{2-hydroxyalkyl} | 0.92 | 0.84 | 0.65 |
| A | 10% C12- | Transmission [%] | 60 | 68 | 75 |
| | C16 APG | γ = 2.55 s⁻¹ [mPas] | 7540 | 5910 | 6160 |
| | 0.5% SD | | | | |
| B | 10% C12- | Transmission [%] | 92 | 94 | 98 |
| | C16 APG | γ = 2.55 s⁻¹ [mPas] | 32800 | 21300 | 22500 |
| | 2.25% SLES | | | | |
| | 1% SD | | | | |

TABLE 4

| | | | Methyl | Methylcarboxy- |
|---|---|---|---|---|
| | | | starch | methyl starch |
| | | Ex. | 8 | 9 |
| | | DS_{carboxymethyl} | / | 0.43 |
| | | DS_{methyl} | 0.73 | 0.73 |
| A | 10% C12- | Transmission [%] | 0.9 | insoluble |
| | C16 APG | γ = 2.55 s⁻¹ [mPas] | 3610 | insoluble |
| | 1% SD | | | |
| B | 10% C12- | Transmission [%] | 92 | insoluble |
| | C16 APG | γ = 2.55 s⁻¹ [mPas] | 22700 | insoluble |
| | 2.25% SLES | | | |
| | 1% SD | | | |

1. A method for the production of a viscosity regulator,
wherein firstly starch is modified hydrophobically by alkylation and/or hydroxyalkylation and subsequently is carboxymethylated by conversion with halogenated acid.

2. The method according to claim 1,
wherein the alkylation is effected with at least one linear or branched C₁-C₂₀ alkylhalogenide.

3. The method according to claim 2,
wherein the alkylation is effected with at least one linear or branched C₁-C₁₂ alkylhalogenide.

4. The method according to claim 1,
wherein the alkylation is implemented at temperatures in the range of 40° C. to 100° C.

5. The method according to claim 1,
wherein the degree of substitution (DS) with respect to alkyl substituents is in the range of 0.1 to 1.2.

6. The method according to claim 1,
wherein the degree of substitution (DS) with respect to carboxymethyl substituents is in the range of ≧0 to 1.2.

7. The method according to claim 1,
wherein the hydroxyalkylation is effected with a linear or branched epoxyalkane, the alkane having a chain length of 2 to 20 C-atoms.

8. The method according to claim 7,
wherein the hydroxyalkylation is effected with a linear or branched epoxyalkane, the alkane having a chain length of 6 to 12 C-atoms.

9. The method according to claim 7,
wherein the hydroxyalkylation is implemented at temperatures in the range of 80° C. to 160° C.

10. The method according to claim 7,
wherein the degree of substitution (DS) with respect to the hydroxyalkyl substituents is in the range of 0.1 to 1.2.

11. The method according to claim 7,
wherein the degree of substitution (DS) of the carboxymethyl substituents is in the range of 0.5 to 1.6.

12. The method according to claim 1,
wherein the halogenated acid is selected from the group consisting of monochloroacetic acid, monochloropropionic acid, chloromalonic acid and mixtures thereof.

13. The method according to claim 1,
wherein the starch is selected from the group consisting of potato-, wheat-, rice-, maize-, barley-, tapioca starch and mixtures thereof.

14. A viscosity regulator made according to the method of claim 1.

15. A method of thickening a surfactant-containing preparation for cosmetic or pharmaceutical application comprising adding a viscosity regulator made in accordance with claim 14.

16. The method according to claim 15,
wherein the preparation contains sodium lauryl ether sulphate.

17. The method according to claim 16,
wherein the preparation contains 0.5 to 3% by weight sodium lauryl ether sulphate.

18. An oil-in-water emulsion containing an oil component, a lipophilic or hydrophilic active ingredient and at least one viscosity regulator made in accordance with claim 14 as an emulsifier.

19. The emulsion according to claim 18,
wherein the emulsion contains from 0.1 to 5.0% by weight of the viscosity regulator.

20. The emulsion according to claim 18,
wherein the emulsion is aqueous and contains alkylpolyglycosides.

21. The emulsion according to claim 20,
wherein the emulsion contains 0.5 to 20% by weight alkylpolyglycosides.

22. The emulsion according to claim 18,
wherein the emulsion is present in the form of a cream or lotion.

23. The emulsion according to claim 18,
wherein the emulsion is free of ionic or hydrophilic emulsifiers.

24. The emulsion according to claim 18,
wherein the lipophilic active ingredient is selected from the group comprising vitamins, oil-soluble UV filters, bisabolol, fragrances and mixtures thereof.

25. The emulsion according to claim 18,
wherein the hydrophilic active ingredient is selected from the group consisting of polyols, urea, plant extracts, vitamins, self-tanning compounds, UV filters or mixtures thereof.

26. The emulsion according to claim 18,
wherein the oil component is selected from the group consisting of vegetable oils, paraffin oils, di-n-alkylether, fatty acids, fatty alcohols, ester oils, natural and synthetic waxes, silicone compounds and mixtures thereof.

27. The emulsion according to claim 18,
wherein the emulsion contains additives selected from the group consisting of preservatives, solvents, antioxidants, fillers, hydrocolloid formers, pigments, chelating agents, pH regulators and mixtures thereof.

28. A cosmetic or pharmaceutical preparation comprising the emulsion according to claim 18.
